(19) United States
(12) Patent Application Publication
(10) Pub. No.: US 2008/0312532 A1
(43) Pub. Date: Dec. 18, 2008

(54) System and Method for Interacting With a Cell or Tissue in a Body

(75) Inventors: Liesbeth Van Pieterson, Heeze (NL); Bernardus Hendrikus Wilhelmus Hendriks, Eindhoven (NL); Gerhardus Wilhelmus Lucassen, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/093,720

(22) PCT Filed: Nov. 14, 2006
(86) PCT No.: PCT/IB2006/054241

§ 371 Date: Aug. 20, 2008

Related U.S. Application Data
(60) Provisional application No. 60/738,238, filed on Nov. 18, 2005.

(51) Int. Cl. A61B 6/00; A61B 1/04

(52) U.S. Cl. 600/431; 600/476; 600/160

(57) ABSTRACT

A system (10, 100, 300, 400) is provided for identifying certain targeted cells or tissues in a body. The system (10, 100, 300, 400) preferably includes at least one ingestible capsule that accommodates or supports (20) at least one of an illuminating module (30, 114, 322, 422, 452), a detecting module (40, 124, 324, 424, 434, 454), an imaging module (50, 326, 432, 456), a control module (60), or a reservoir (70, 132, 312, 412, 442) suitable for retaining and releasing one or more nanoshells. A method (200) is also provided for employing the system (10, 100, 300, 400) to detect, image or otherwise interact with diseased or abnormal cells and/or tissues in a body. The system (10, 100, 300, 400) and method (200) provided allow for improved diagnosing, treating and imaging techniques and for increased sensitivity and specificity in efficiently distinguishing targeted diseased or abnormal cells or tissue from surrounding healthy cells or tissue.

The present disclosure is related to U.S. Provisional Patent Application No. 60/644,540, entitled “Electronicially Controlled Capsule For Releasing Radiation”, and filed Jan. 18, 2005, U.S. Provisional Patent Application No. 60/644,539, entitled “Electronicially Controlled Capsule”, and filed Jan. 18, 2005, U.S. Provisional Patent Application No. 60/644,538, entitled “Electronicially Controlled Ingestible Capsule”, and filed Jan. 18, 2005, U.S. Provisional Patent Application No. 60/644,518, entitled “System And Method For Controlling Traversal Of An Ingested Capsule”, and filed Jan. 18, 2005, U.S. Provisional Patent Application No. 60/606,276, entitled “Electronically Controlled Pill And System For Delivering At Least One Medicament”, and filed Sep. 1, 2004, and U.S. Provisional Patent Application No. 60/605,364, entitled “Electronically And Remotely Controlled Pill And System For Delivering At Least One Medicament”, and filed Aug. 27, 2004, with each of the foregoing references being assigned to the Assignee of the present disclosure and hereby being expressly incorporated by reference as part hereof.

The present disclosure is directed generally to identifying certain targeted cells and/or tissues in a body, and more particularly to a system and method for detecting and imaging or otherwise interacting with diseased or abnormal cells and/or tissues in a body using a capsule system.

Ingestible capsules having imaging capabilities are known in the art. For example, U.S. Pat. No. 6,240,312 to Alfano et al., issued May 29, 2001, which patent is hereby incorporated herein by reference, reads on an ingestible capsule system having a camera that acquires diagnostic images as it traverses the alimentary tract. In addition, U.S. Pat. No. 6,324,418 to Crowley et al., issued Nov. 27, 2001, which patent is also hereby incorporated herein by reference, reads, at least in part, on a capsule system for performing tissue spectroscopy.

Notwithstanding the foregoing, and other like prior art capsule systems providing clear benefits, such systems still have significant shortcomings associated therewith. For example, as discussed in Int. App. No. PCT/US2003/024163 (Pub. No. WO2004/032621 A2) to Madar et al., published Apr. 22, 2004, which application is hereby incorporated herein by reference, existing capsule systems often have difficulty distinguishing between diseased or abnormal tissue and healthy tissue having similar characteristics. Madar et al., attempt to address this shortcoming by utilizing an exogenous fluorescent-labeled probe that binds to or is internalized by certain cells. This probe can be introduced into an abnormal cell or tissue so as to emit a distinguishing fluorescent signal. Once introduced, the fluorescent signal can be detected via, for example, a detecting capsule.

However, as discussed via U.S. Patent Application Publication No. 2002/0103517 to West et al., published Aug. 1, 2002, which application is also hereby incorporated herein by reference, these prior art fluorescent probes also have certain shortcomings associated therewith. For example, not all probes are effectively transferred to the desired or intended location. Oftentimes, a large fraction of the probes do not end up at the appropriate location or have a low uptake rate, either of which conditions can make it difficult, when recording a luminescence spectrum, to separate out the desired signal from the background signal. Also, as excitation in the visible region of the spectrum is usually necessary, at the required wavelengths, many constituents of human cells or tissue tend to auto-fluoresce as well. The auto-fluoresce of other tissue-types is clearly an undesired effect, which can make it difficult to resolve the desired luminescence of the probes at the intended location.

The foregoing issues are compounded by a number of prior art capsule systems in that many of the known systems have a limited ability to view an entire targeted area. That is, many conventional capsule systems provide for only one directional viewing over a short or limited period of time, thus making effective identification, imaging and/or treatment of a targeted cell or tissue difficult and inefficient.

In an effort to address certain of the above-identified shortcomings, biocompatible metal nanoparticles or nanoshell composites have been constructed in a manner to allow for a choice of core material, core dimensions, and core geometry independent of those criteria for the shell material. These nanoshells are relatively homogeneous structures that, inter alia, (i) do not have to rely on suspension in a particular medium in order to exhibit their desired absorption characteristics, and (ii) can be linked to antibodies that recognize diseased or abnormal cells or tissue. Moreover, these nanoshells, with a luminescent ion (e.g., Pr⁺³, Er⁺³, and/or Nd⁺³) in the dielectric material thereof, for instance, effectively allow for excitation in the near-infrared region of the spectrum, at which excitation wavelength, cells or tissue inside the human body generate essentially no visible emission.

Thus, nanoshells effectively address at least certain aspects of the above-noted shortcomings of the prior art, and when utilized in combination with an appropriate capsule system such as that which is provided via the present disclosure, these nanoshells beneficially allow for improved diagnosing, treating and imaging techniques and for increased sensitivity and specificity in efficiently distinguishing targeted diseased or abnormal cells or tissue from surrounding healthy cells or tissue.

According to a beneficial feature of present disclosure, a system is provided in which one or more supports are used to accommodate one or more illuminating modules, one or more detecting modules, one or more imaging modules, and one or more control modules. Each illuminating module being suitable to illuminate a targeted cell or tissue, each detecting module being suitable to detect a targeted cell or tissue, each imaging module being suitable to image a targeted cell or tissue, and each control module being suitable to control or influence any illuminating module, any detecting module, and/or any imaging module. Advantageously, the system of the present disclosure provides for, among other things, improved bioscopy imaging. Another advantage provided by the system of the present disclosure is found in that the system, in at least one aspect, provides for multidirectional detecting and imaging, as well as viewing, of a targeted cell or tissue for an extended period of time.

In an illustrative aspect of the present disclosure, the system includes a single support preferably including at least one illuminating module, at least one detecting module, at least one imaging module, and at least one control module operatively associated with each of the respective modules so as to influence the operation thereof. Advantageously, the single support may also include at least one reservoir for retaining one or more nanoshells, which nanoshells may be of a single type or of two or more differing types. The single support may further advantageously include at least one delivery mechanism operatively associated with at least one reservoir so as to allow for the delivery or release of one or more nanoshells as desired.

A method according to a beneficial feature of the present disclosure includes the steps of: (i) delivering one or more nanoshells to one or more areas in a body; (ii) detecting a targeted cell or tissue identified via the delivered nanoshells; and (iii) when the targeted cell or tissue is detected, then at least one of determining the precise location of the targeted cell or tissue, imaging the targeted cell or tissue, or delivering one or more additional nanoshells to the targeted cell or tissue. In an illustrative aspect of the present disclosure, the delivering step may be accomplished via an ingestible support. In another illustrative aspect of the present disclosure, the delivering step is accomplished via a first support and the detecting and imaging steps are accomplished via a second support. In still another illustrative aspect of the present disclosure, each step (i), (ii) and (iii) is individually accomplished via different ingestible supports. As will be readily apparent to those skilled in the pertinent art from the present disclosure, in each identified illustrative aspect, a support may include any of a variety of module combinations, and further may include modules not specifically identified and discussed via the present disclosure and that may be capable of performing any of a variety of functions.

In another illustrative aspect, the system of the present disclosure may include two supports, a first support and a second support. The first support preferably includes at least one reservoir for retaining one or more nanoshells of one or more type, and at least one delivery mechanism operatively associated with at least one reservoir so as to allow for the delivery or release of one or more nanoshells as desired. The second support preferably includes at least one illuminating module, at least one detecting module, and at least one imaging module. Advantageously, either or both of the two supports may also include a control module. Such control module(s) may beneficially be operatively connected to any one or more of the identified system modules and/or delivery mechanism(s) in any of a variety of combinations.

In yet another illustrative aspect, the system of the present disclosure may include three or more supports, with each support including at least one system module. For example, a first support may include a reservoir and a delivery mechanism operatively associated therewith, a second support may include an illuminating module, a third support may include a detecting module, a fourth support may include an imaging module, a fifth support may include an additional reservoir and delivery mechanism and/or any combination of other modules. In other aspects of the present disclosure, each support may include any of a variety of module combinations, and may include modules not specifically identified and discussed via the present disclosure and that may be capable of performing any of a variety of functions.

Additional advantageous features, aspects and/or functions relating to the present disclosure will be apparent from the detailed description which follows, particularly when reviewed together with the appended figures, which figures are referenced to assist those of ordinary skill in the art to which the subject matter of the present disclosure appertains to better understand the illustrative examples of the present disclosure, wherein:

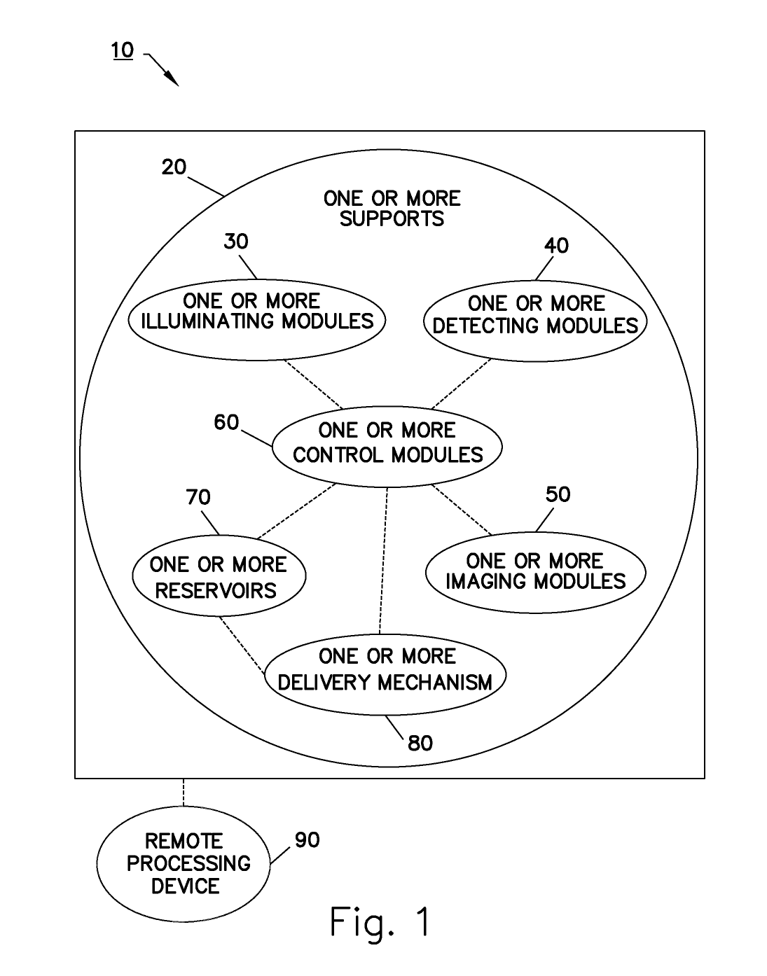

FIG. 1 is a schematic representation of a system in accordance with an illustrative aspect of the present disclosure;

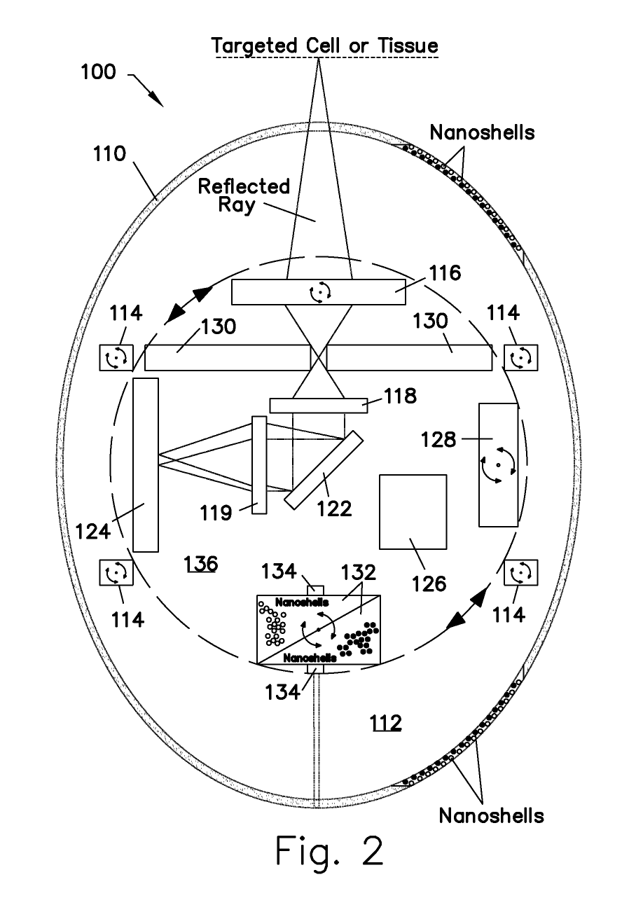

FIG. 2 is a block diagram showing an exemplary support in accordance with an illustrative aspect of the present disclosure;

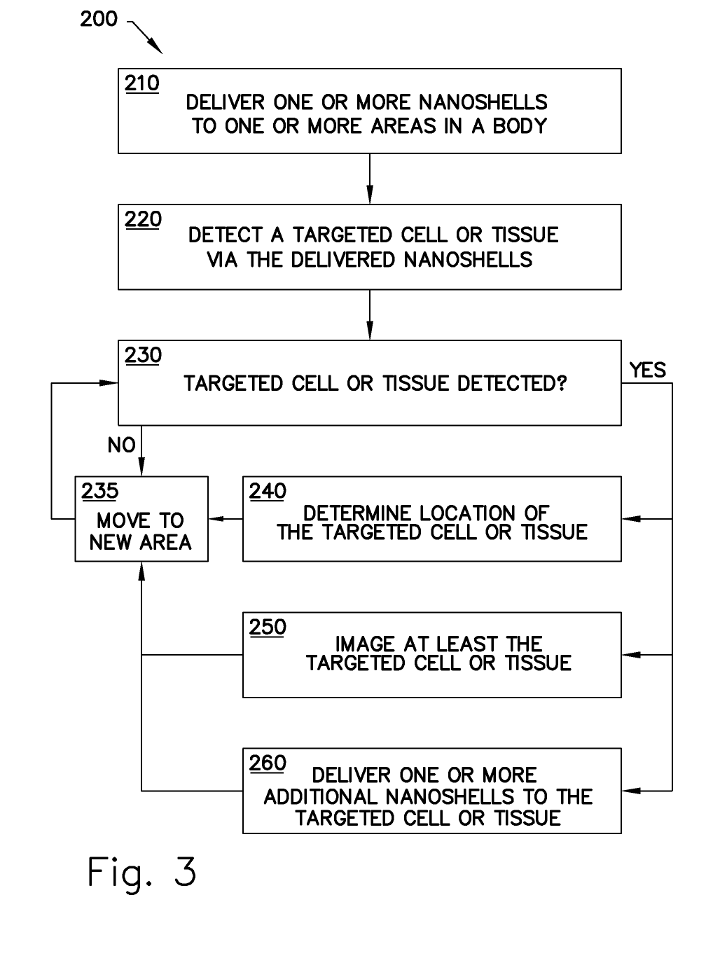

FIG. 3 is a flow diagram of a method in accordance with a beneficial aspect of the present disclosure;

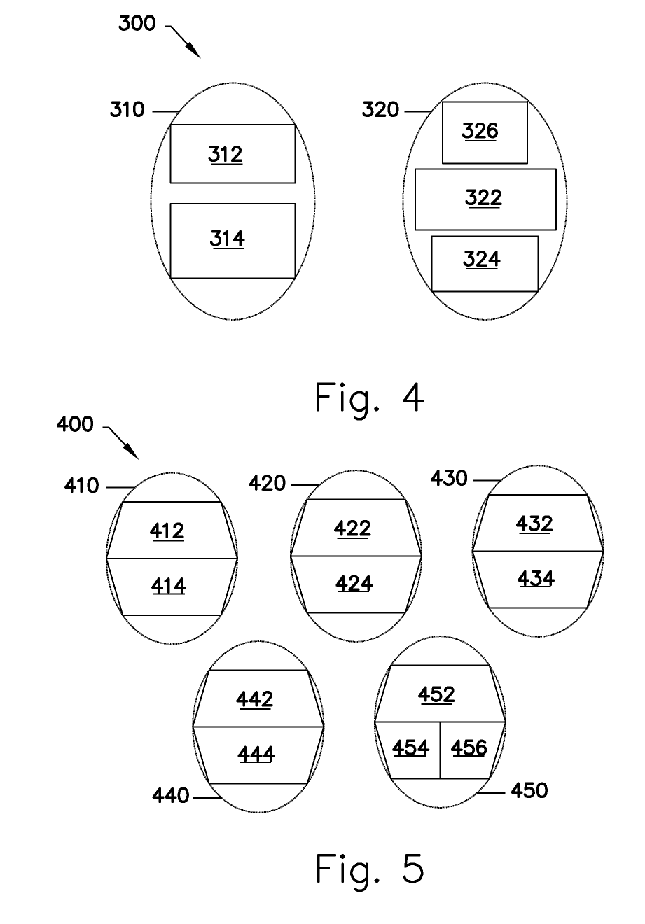

FIG. 4 is a schematic representation of a system in accordance with another illustrative aspect of the present disclosure; and

FIG. 5 is a schematic representation of a system in accordance with a further illustrative aspect of the present disclosure.

With reference to the drawings, it should be understood that notwithstanding the following detailed description of the various examples and/or aspects of the present disclosure referring to the drawings which form a part hereof, other additional and/or alternative examples, aspects and/or features may equally be used without departing from the scope of the present disclosure as the advantageous features of the present disclosure may be employed in any of a variety of applications including, for example, treating a targeted cell or tissue.

With initial reference to FIG. 1, there is shown an exemplary system 10 in accordance with an illustrative aspect of the present disclosure. As shown, such system 10 preferably includes at least one support 20, at least one illuminating module 30, at least one detecting module 40, at least one imaging module 50, and at least one control module 60. Further, in at least one aspect of the present disclosure the system 10 may also include at least one reservoir 70 and at least one delivery mechanism 80 operatively associated the reservoir 70. The reservoir 70 and delivery mechanism 80 are preferably suitable to accommodate and deliver or dispense one or more nanoshells as desired.

The support 20, in an illustrative aspect of the present disclosure, can be made from bio-compatibles materials such that the support 20 is biocompatible for at least the amount of time it requires to traverse through the body, or a portion thereof (e.g., the gastrointestinal tract). Further, the support 20 may preferably be made from materials used to fabricate implantable devices, including, for example, pacemaker leads and cardiac prosthesis devices, such as artificial hearts, heart valves, intra-aortic balloons, and ventricular assist devices. Examples of such materials include Pellethane® 2363 polyether urethane series of materials available from Dow Chemical Company, and Elasthane polyether urethane available from the Polymer Technology Group, Inc. Other illustrative materials that might also be appropriate include PurSil® and CarboSil® also available from the Polymer Technology Group, Inc.

The support 20, in different aspects of the present disclosure, can have any of a variety of shapes, sizes, colors, textures and/or any other characteristic or property necessary to accomplish any of a variety of different aesthetic and/or functional purposes consistent with the present disclosure. For example, and without limitation, the support 20 have a micro-porous membrane with holes ranging in size from sub-micron to a few microns in diameter. The membrane can be infused or impregnated with nanoshells, wherein upon stretching of the membrane, such as by some mechanical means, the nanoshells may be released at a controlled rate over a specific area. Alternatively, nanoshells may be coated onto a surface of the support 20 and delivered to a specific site. Pressure, heat, laser light, etc., may facilitate transfer of the nanoshells from the surface of the support 20 to a targeted area in the body. In addition, in yet another aspect of the present disclosure, the support 20 can take the form of an endoscope or the like, which endoscope may include a tip with at least one system module operatively associated therewith. In another aspect, the endoscope support may be used in association with another support so as to accomplish various beneficial operations consistent with the present disclosure (e.g., the endoscope guides the other support to a desired location).

It is noted that those skilled in the art will readily appreciate, from the present disclosure that variations to the support 20 may be made without departing from the spirit and scope of the present disclosure. For example, the support 20 can be substantially transparent or translucent so as to allow light of a desired wavelength to be emitted, via the illuminating module 30, in substantially all directions.

The illuminating module 30, in an illustrative aspect of the present disclosure, is preferably operatively associated with the support 20, and preferably includes at least one light source, such as a light emitting diode (LED), a xenon tube, a laser source, or the like. For example, any of a variety of LEDs that emit light at a variety of different wavelengths with a variety of different wavelength bands may be selected so as to match the excitation of a target cell or tissue. In other aspects of the present disclosure, the light source can have a broad spectrum, such as white light LED. As will be readily apparent to those skilled in the pertinent art from the present disclosure, in still other aspects of the present disclosure, a variety of other light sources can additionally, or alternatively, be used so as to accomplish any of a variety of different functional purposes consistent with the present disclosure.

According to an illustrative aspect of the present disclosure, in operation, the illuminating module 30 emits at least one light or laser beam that impacts and is reflected from a targeted cell or tissue near the support 20. The targeted cell or tissue will preferably have distinct light reflectivity properties, which properties may be inherent to the cell or tissue, or artificially created via some type of marker. The orientation of the illuminating module 30 relative to the ambient environment, in certain aspects of the present disclosure, may be adjustably controlled for aiming the light source in a desired direction. In other aspects of the present disclosure, the illuminating module 30 includes a light source suitable to emit light of a desired wavelength in substantially all directions simultaneously and/or sequentially. For example, the illuminating module 30 may include a light source that is operatively associated with a device or means permitting the light source to incline freely and/or adjustably in any direction and/or to be suspended so that it will remain level when the support 20 is tipped or rotated (e.g., a gimbal). Alternatively, or in addition, as previously indicated, the illuminating module 30 may be operatively associated with at least substantially transparent or translucent support so as to allow the light source to emit light in multi-directions through or about the support 20.

As with the support 20, it is noted that those skilled in the art will readily appreciate, from the present disclosure that variations to the illuminating module 30 may be made without departing from the spirit and scope of the present disclosure. For example, the illuminating module 30 can include a micro-scale, solid-state, semiconductor diode laser system or a flash lamp suitable for use as a surgical source to ablate, weld or otherwise affect cells or tissue.

The detecting module 40, in an illustrative aspect of the present disclosure, can be operatively associated with the support 20, and preferably includes at least one photo detector for sensing incident light and generating a corresponding sensing signal, which preferably includes a minimal number of photo detectors, such as one or two rows of photo detectors or one photo detector. The detecting module 40 preferably detects reflected light incident on the one or more photo detectors thereof and preferably generates a corresponding light sensing signal. The detecting module 40 may be configured for outputting a digital signal corresponding to the light sensing signal.

The detecting module 40, in an aspect of the present disclosure, preferably includes at least one or more micro-scale narrow band filters, color filters, and/or any other component suitable for manipulating received light and/or the spectral characteristics (e.g., intensity, distribution, etc.) thereof so as to facilitate distinguishing a targeted cell or tissue from adjacent cells or tissues. For example, wide-band dielectric filters can be used to separate various colors emitted or absorbed. The detecting module 40 may also use MEMS circuitry equivalent to circuitry found in optical code detectors, such as laser-based optical code readers or imaging-based optical code readers.

Those skilled in the art will readily appreciate, from the present disclosure that variations to the detecting module 40 may be made without departing from the spirit and scope of the present disclosure. For example, the detecting module 40 may consist of a CCD suitable to detect fluorescent signals. Further, the detecting module 40 may include one or more detecting sensors that are operatively associated with a device or means permitting the sensors to incline freely and/or adjustably in any direction and/or to be suspended so that it will remain level when the support 20 is tipped or rotated (e.g., a gimbal). Alternatively, or in addition, the detecting module 40 may be operatively associated with at least substantially transparent or translucent support so as to allow for multi-directional sensing or detection of a desired wavelength through or about the support 20.

The imaging module 50, in an illustrative aspect of the present disclosure, is preferably operatively associated with the support 20, and preferably includes at least one camera (e.g., a micro-video CCD) suitable for collecting and/or transmitting images, so that a person or a computer-aided detection system (e.g., CAD, CADx, etc.) can detect changes in the texture of a targeted cell or tissue. This texture detection feature may also be used to determine where in a body the targeted cell or tissue is located. For example, changes in texture may be correlated with different sections of the alimentary tract, such as the esophagus, stomach, duodenum function between stomach and small intestine), cecum function of small and large intestine), and rectum. The images acquired via the imaging module 50, in a preferred aspect of the present disclosure, can be analyzed by a remote processing device 90, for example, in real time and/or at a later time as desired via appropriate transmission means (e.g., a wireless type, a wire type, etc.). Thus, an area targeted for a real-time procedure/treatment, a subsequent procedure/treatment, or both, may, in an aspect of the present disclosure, be better determined based on the acquired images.

Those skilled in the art will readily appreciate, from the present disclosure that variations to the imaging module 50 may be made without departing from the spirit and scope of the present disclosure. For example, the imaging module 50 may include one or more cameras that are operatively associated with a device or means permitting the cameras to incline freely and/or adjustably in any direction and/or to be suspended so that it will remain level when the support 20 is tipped or rotated (e.g., a gimbal). Alternatively, or in addition, the imaging module 50 may be operatively associated with at least substantially transparent or translucent support so as to allow for multi-directional imaging through or about the support 20.

The control module 60, in an illustrative aspect of the present disclosure, is preferably operatively associated with the illuminating module 30, the detecting module 40, and/or the imaging module 50 so as to influence or control the operation thereof. In addition, in certain aspects of the present disclosure, the control module 60 may also be operatively associated with the reservoir 70 and/or delivery mechanism 80. The control module 60, in an aspect of the present disclosure, preferably includes both processing and communication means for interacting with and/or influencing the illuminating module 30, the detecting module 40, and/or the imaging module 50 (i.e., system module(s)). In another aspect of the present disclosure, the control module 60 includes communication means for communicating with remote processing means (e.g., a computer). For example, the control module 60 may communicate with remote processing means via an antenna and transmitter/receiver device. Alternatively, or additionally, communication between the control module 60 and remote processing means may be provided via a transducer and an external ultrasound probe also having a transducer. As will be readily understood by those skilled in the art any of a variety of other communication techniques may equally be employed without departing from the spirit or scope of the present disclosure.

The control module 60, in a preferred aspect of the present disclosure, can communicate with the various system module(s) so as to control, inter alia, the activation and/or deactivation thereof, and/or the orientation or position thereof. Further, the control module 60 may also control, either directly or indirectly, the illumination intensity, wavelength, distribution, and/or pattern provided via the illuminating module 30, the detection sensitivity and/or specificity provided via the detecting module 40, and/or the image quality and/or production provided via the imaging module 50. In an aspect of the present disclosure, communication by and between the control module 60 and any one or more of the system module(s) can be either via wire or wireless, such as, for example, via optical signals.

As will be readily apparent to those skilled in the pertinent art from the present disclosure, variations to the control module 60 may be made without departing from the spirit and scope of the present disclosure. For example, the control module 60, in certain aspects of the present disclosure, can be a microprocessor specifically designed for the system 10, such as an application specific integrated circuit (ASIC). In other aspects, the control module 60 is a general-purpose signal processing or computer chip programmed to function in a particular way (e.g., to determine when a particular condition is satisfied).

The reservoir 70, in an illustrative aspect of the present disclosure, is preferably operatively associated with the support 20, and preferably suitable to accommodate a supply of nanoshells of at least one type and providing or facilitating at least one effect (e.g., detecting, imaging, therapeutic, diagnosing, etc.). In certain aspects of the present disclosure, the reservoir 70 can be integral with the surface of the support 20. In other aspects, the reservoir 70 can be internal to the support 20. For example, as shown in FIG. 2, one or more reservoirs can be accommodated or defined within the support, while others are integral with a wall thereof. Each reservoir is preferably operatively associated with one or more release or delivery mechanisms, such as, for example, mechanical means (e.g., valves, pumps, pistons, etc.), or electrochemical means (e.g., a chemical reaction, etc.), or some combination thereof.

In still other aspects of the present disclosure, one or more reservoirs 70 can be advantageously positioned (e.g., at an edge, proximate a surface, or other location) with respect to other modules (e.g., illuminating, detecting, and/or imaging modules) associated with the pertinent portion/module of the support 20 so that at least some nanoshells contained by the reservoir can be delivered to a targeted cell or tissue prior to, or simultaneously with, the support 20 passing by or over the targeted cell or tissue. For example, a reservoir 70 can be located at a forward edge of the support 20 so that nanoshells released therefrom and delivered to a targeted cell or tissue in time for the nanoshells to effect the targeted cell or tissue, as desired, prior to, or at the same time as, that portion of the support 20 which accommodates the illuminating module 30, detecting module 40, and/or the imaging module 50 passes over or by the targeted cell or tissue.

In yet another aspect of the present disclosure, at least two reservoirs 70 are operatively associated with the support 20 such that one reservoir can deliver at least some nanoshells of a first type to a targeted cell or tissue prior to the support 20 passing by or over such targeted cell or tissue, and another reservoir can deliver at least some nanoshells of a second type to the same, or a different, targeted cell or tissue prior to, or simultaneously with, the support 20 passing by or over such targeted cell or tissue. Further, it is noted that nanoshells of a first type can be delivered so as to have a first effect on the targeted cell or tissue (or adjacent tissue), with nanoshells of a second type being deliverable so as to have a second effect on the targeted cell or tissue (or adjacent tissue). The first and second effects provided by the respective first and second nanoshells can be equivalent, identical, different, assorted, cumulative, cooperative, interactive, or otherwise related in order to accomplish any of a variety of different purposes (e.g., detecting, imaging, diagnosing, treating, etc.) with respect to the targeted cell or tissue and consistent with the present disclosure.

As will be readily apparent to those skilled in the pertinent art from the present disclosure, variations to the reservoir 70 may be made without departing from the spirit and scope of the present disclosure. For example, one or more reservoirs 70, in certain aspects of the present disclosure, can be deformable so that pressure exerted on such a reservoir causes or facilitates delivery of nanoshells through, for example, at least one opening in the reservoir 70. An example of suitable pressure means might be an artificial muscle formed of a polymer that controllably expands or contracts in response to an applied electrical signal so as to apply pressure to the reservoir and/or the stored nanoshells.

The delivery mechanism 80, in an illustrative aspect of the present disclosure, is preferably operatively associated with both the support 20 and the reservoir 70. However, in other aspects, this need not be so. For example, in certain aspects of the present disclosure, the support 20 is itself suitable to accommodate one or more nanoshells directly (e.g., via surface coating or infusion) and to thereby serve, at least in part, as both the delivery mechanism and reservoir.

In another aspect of the present disclosure in which one or more reservoirs are accommodated or defined within the support (e.g., as shown in FIG. 2), each reservoir can have a delivery mechanism, such as a valve, associated therewith. Each valve is preferably operatively associated with the control module 60 so as to be controlled thereby (e.g., via control signals) such that one or more nanoshells can be released or delivered to a targeted cell or tissue as desired (e.g., intermittently, timely, judiciously, or otherwise).

In still another aspect of the present disclosure, the delivery mechanism 80 can take the form of a pressure element, including, for example, a displaceable and/or expandable member (e.g., a spring piston) which is suitable to exert pressure on at least one reservoir for displacing any contents (e.g., nanoshells) thereof and/or to cause such contents to exit the reservoir. Controlling a valve, or the like, also included as part of the delivery mechanism may additionally control dispensing of the reservoir contents, such that, upon, or after, exiting the reservoir, the contents are either directly or indirectly delivered to a targeted cell or tissue in a body.

Those skilled in the pertinent art, from the present disclosure, will readily appreciate variations to the delivery mechanism 80 may be made without departing from the spirit and scope of the present disclosure. For example, the delivery mechanism 80 might be an artificial muscle formed of a polymer that controllably expands or contracts in response to an applied electrical signal so as to apply controlled pressure to the reservoir and/or the contents thereof and thereby cause an effect (e.g., mixing, dispensing, etc.) with respect to the contents as desired.

Having identified and discussed various beneficial aspects and features associated with the system of the present disclosure, with reference now to FIG. 2, an example of how such system might be characterized is illustrated. As shown, an ingestible capsule that may be employed as a support in accordance with the present disclosure is indicated generally by the reference numeral 100. The ingestible capsule 100 preferably has a wall 110 that defines a cavity 112 suitable to accommodate one or more system elements. For instance, as shown, the cavity 112 can accommodate illuminator(s) 114, a lens set (e.g., capturing lens 116, transforming lens 118, projecting lens 119), grating(s) 122, detector(s) 124, controller(s) 126, imager(s) 128, sensor(s) 130 and/or reservoir(s) 132 with associated delivery mechanism(s) 134. Thus, in an aspect of the present disclosure, the imaging lens 116 is preferably suitable to form an image on the image sensor 130. That is, via a hole in the center of the image sensor 130, for example, light emitted or reflected from a cell or tissue at a zero field of view can pass through the image sensor 130 so as to interact with the lens set, the grating and/or detector. Hence, the detector, in one aspect of the present disclosure, can be a spectrometer suitable to receive light deflected from a grating, which light is transmitted and/or received at different angles depending on its wavelength so that it is imaged on the spectrometer, via projecting lens 119, for example, at different positions depending on its wavelength, which different positions allow for the spectral content of the light to be resolved via various elements of the spectrometer.

In an aspect of the present example, the illuminator(s) 114 can be adjustable in that they can move or pivot and/or provide light at different intensities. In another aspect of the present example, the lens set, or a least certain lenses thereof, are adjustable as well. In a further aspect of the present example, the reservoir(s) 132 can likewise be adjustable. Moreover, in a preferred aspect of the present example, each system element is secured to a gimbal-like structure 136 so as to define a composite assembly capable of moving with at least one, and preferably three (e.g., roll, pitch, yaw), degrees of freedom within the cavity 112. For example, the gimbal-like structure 136 (w/system elements secured thereto) might float within the cavity 112, via a fluid (gas or liquid), and/or be capable of inclining freely and/or adjustably in any direction, and/or being suspended so that it will remain level when the capsule 100 is tipped or rotated, and/or moving relative to the wall 110 of the cavity 112 so as to facilitate various imaging, detecting, diagnosing and/or treating operations.

Those skilled in the pertinent art, from the present disclosure, will readily appreciate variations to the capsule 100 may be made without departing from the spirit and scope of the present disclosure. For example, the capsule 100 might include one or more color filters in place of grating(s) 122, or the capturing lens 116 thereof might be a fluid focus lens as described in PCT International Publication No. WO2004051323, entitled “Apparatus For Forming Variable Fluid Miniscus Configurations”, and published Jun. 17, 2004, which reference is hereby incorporated herein by reference.

Turning to FIG. 3, there is shown a flow diagram illustrating a method according to yet another aspect of the present disclosure. Although steps are shown in FIG. 3 in a particular arrangement for purposes of illustration, in other aspects of the present disclosure, the steps may be performed in a different order or in an overlapping manner. For example, in certain aspects of the present disclosure, step 210 can be performed after step 220 and in other aspects, step 210 can be omitted, as in the case when a targeted cell or tissue is detected via some means other than by nanoshells.

In step 210 one or more nanoshells are delivered to one or more areas in a body. Any method for delivering the nanoshells may be used including, injection into the blood stream, injection into tissue, oral ingestion, and/or direct point delivery, among others. In a preferred aspect of the present disclosure, the nanoshells can be delivered via an ingestible capsule such as, for example, shown via FIG. 2. As shown, the nanoshells can be accommodated via a reservoir internal to the ingestible capsule. Alternatively, or in addition, one or more nanoshells can be accommodated via a wall and/or surface of the ingestible capsule.

In step 220, according to a preferred aspect of the present disclosure, a targeted cell or tissue (e.g., cancerous cell or tissue) can be detected via at least one effect provided by the delivered nanoshells. In other aspects, the targeted cell or tissue can be detected via other means, such as, for example, by way of a fluorescence probe and/or imaging technology (e.g., x-ray, CT, MR, etc.). If the targeted cell or tissue is detected, then control passes to at least one of steps 240, 250 or 260. If the targeted cell or tissue is not detected, then control passes to step 235. In certain aspects of the present disclosure, step 230 can include performing other processes or functions. For example, step 230 can include obtaining measurements (e.g., measure the level of a detectable characteristic of the effect provided via the nanoshells), or identifying particular characteristics associated with the cell or tissue directly or with the effects provided via the nanoshells in combination with the cell or tissue.

In step 235, the detecting means (e.g., ingestible capsule 100 with or without nanoshells) moves to another area in the body. Relocating the detecting means can be accomplished by any of a variety of processes (e.g., self propulsion, peristalsis, etc.). In certain aspects of the present disclosure, the detecting means can be tracked or monitored via signal transmission (e.g., radio frequency (rf)) to a remote monitoring or observation unit.

In step 240, according to an aspect of the present disclosure, the precise location of the cell or tissue, once detected, can be determined via any of a variety of techniques. For example, an effect (e.g., light, heat, vibration, etc.) provided via the nanoshells delivered to the targeted cell or tissue is such that it is easily identified relative to the rest of the body via external and/or internal detecting means (e.g., sensors, imagers and the like).

In step 250, according to another aspect of the present disclosure, the targeted cell or tissue can be imaged by way of an internal imager (e.g., via ingestible capsule 100 or other suitable device). In other aspects of the present disclosure, the targeted cell or tissue is imaged by way of an external imaging device known (e.g., x-ray, CT, MR, etc.) or which may become known.

In step 260, according to still another aspect of the present disclosure, one or more additional nanoshells can be provided or delivered to the targeted cell or tissue. This may be beneficial in that providing additional nanoshells to the targeted cell or tissue may improve or enhance the effect provided thereby. Also, in certain aspects of the present disclosure, the additional nanoshells can differ from the previously provided nanoshells. For example, nanoshells of a first type can be delivered so as to have a first effect on a targeted cell or tissue, while nanoshells of a second type can be delivered (subsequently or contemporaneously) and can have a second effect on the targeted cell or tissue. The effects provided by the nanoshells can be equivalent, identical, different, assorted, cumulative, cooperative, interactive, or otherwise related so as to accomplish any of a variety of different purposes (e.g., detecting, imaging, diagnosing, treating, etc.) with respect to the targeted cell or tissue.

As will be readily apparent from the present disclosure to those skilled in the pertinent art, variations to the foregoing method may be made without departing from the spirit and scope of the present disclosure. For example, certain steps may be accomplished via two or more ingestible capsules such as discussed herein. In addition, with respect to step 260, for instance, in another aspect of the present disclosure, the additional nanoshells can be replaced by a medicament (e.g., a cancer treating drug). That is, after one or more nanoshells have been used to identify a targeted cell or tissue, a particular medicament can be delivered to the targeted cell or tissue so as to an affect thereon.

With reference to FIG. 4, a system according to another illustrative aspect of the present disclosure is indicated generally by reference numeral 300. As shown, the system 300 includes at least two ingestible capsules. A first capsule 310 preferably accommodating a reservoir 312 and a delivery mechanism 314, and a second capsule 320 preferably accommodating an illuminating module 322, a detecting module 324, and an imaging module 326. In other aspects of the present disclosure, the capsules 310, 320 may include other system modules such as any of those discussed herein. For example, either or both capsules 310, 320 may include a control module so as to influence the operation of at least one other system module. At least one advantage of system 300 is found in that the capsule 310 can have more capacity to accommodate a larger number of nanoshells then, say, for example, capsule 320, which capsule accommodates other system modules that take up space. It is noted that system 300 is well suited for use in performing method 200 of FIG. 3. For example, according to an illustrative aspect of the present disclosure, capsule 310 can be used to accomplish step 210, while capsule 320 can be used to perform at least one of steps 220, 240, 250 or 260. It is further noted that from the present disclosure, those skilled in the pertinent art will readily appreciate various applications or uses for system 300, and that any such application or use should be considered to fall within the spirit and scope of the present disclosure.

Turning to FIG. 5, a system according to still another illustrative aspect of the present disclosure is indicated generally by reference numeral 400. As shown, the system 400 includes three or more ingestible capsules with each being suitable to accommodate at least one system module. For example, a first capsule 410 can include a reservoir 412 and a delivery mechanism 414, a second capsule 420 can include an illuminating module 422, a detecting module 424, or both, a third capsule 430 can include an imaging module 432, a detecting module 434, or both, a fourth capsule 440 can include an additional reservoir 442 and an additional delivery mechanism 444, and/or a fifth capsule 450 can include an additional illuminating module 452 and at least one of an additional detecting module 454, or an additional imaging module 456. In other aspects of the present disclosure, the capsules 410, 420, 430, 440, 450 may include any other system module such as those previously discussed herein. For example, any such capsule may include a control module so as to influence the operation of at least one other system module. At least one advantage of system 400 is found in that the capsules 410, 440, like capsule 310, can have capacity to accommodate a larger number of nanoshells then, say, for example, capsules 420, 430, which capsules accommodate other system modules that take up space. It is noted that system 400, like system 300, is also well suited for use in performing method 200 of FIG. 3. For example, according to an illustrative aspect of the present disclosure, capsules 410 and 440 can be used to accomplish steps 210 and 260, respectively, while capsules 420, 430, and 450 can be used to perform any of steps 220, 240, 250 or 260. It is also noted that from the present disclosure, those skilled in the pertinent art will readily appreciate various applications or uses for system 400 not specifically discussed, and that any such application or use should be considered to fall within the spirit and scope of the present disclosure.

As many aspects, features and advantages identified and described herein are apparent from the foregoing detailed discussion, it is intended by the appended claims to cover all such aspects, features and advantages that fall within the spirit and scope of the present disclosure. Further, since numerous modifications and changes will readily occur to those skilled in the art, it is not desired to limit the scope of the present disclosure to the exact construction and operation illustrated and described, and accordingly all suitable modifications and equivalents may be resorted to as falling within the present disclosures scope. Thus, the exemplary aspects and/or features described herein are merely illustrative and the present disclosure specifically encompasses alternative and/or modified aspects and/or features of that which has been disclosed.

1. A system (10, 100, 300, 400) comprising:
a support (20);
an illuminating module (30, 114, 322, 422, 452) for illuminating a targeted cell or tissue;
a detecting module (40, 124, 324, 424, 434, 454) for detecting the targeted cell or tissue;
an imaging module (50, 326, 432, 456) for imaging the targeted cell or tissue; and
a reservoir (70, 132, 312, 412, 442) for accommodating one or more nanoshells.

2. The system (10, 100, 300, 400) of claim 1, wherein the support (20) is an ingestible capsule.

3. The system (10, 100, 300, 400) of claim 2, wherein the capsule is substantially transparent so as to allow light of a desired wavelength to pass therethrough.

4. The system (10, 100, 300, 400) of claim 2, wherein at least one of the illuminating module (30, 114, 322, 422, 452), the detecting module (40, 124, 324, 424, 434, 454), or the imaging module (50, 326, 432, 456) is accommodated by the capsule so as to be capable of moving with at least one degree of freedom.

5. The system (10, 100, 300, 400) of claim 2, wherein the reservoir (70, 132, 312, 412, 442) is part of the capsule and capable of retaining and releasing nanoshells of at least one type.

6. The system (10, 100, 300, 400) of claim 2, further comprising a control module (60) for controlling at least one of the illuminating module (30, 114, 322, 422, 452), the detecting module (40, 124, 324, 424, 434, 454), or the imaging module (50, 326, 432, 456).

7. The system (10, 100, 300, 400) of claim 6, wherein the control module (60) also controls the position and orientation of at least one of the illuminating module (30, 114, 322, 422, 452), the detecting module (40, 124, 324, 424, 434, 454), the imaging module (50, 326, 432, 456), or the capsule itself.

8. A method comprising the steps of:
(a) delivering one or more first nanoshells to one or more areas in a body;
(b) detecting a targeted cell or tissue identified via the delivered nanoshells;
and
(c) when the targeted cell or tissue is detected, then at least one of determining the precise location of the targeted cell or tissue, imaging at least the targeted cell or tissue, treating the targeted cell or tissue, or delivering one or more second nanoshells to the targeted cell or tissue.

9. The method of claim 8, wherein step (a) is accomplished via an ingestible support (20).

10. The method of claim 9, wherein steps (b) and (c) are accomplished via another ingestible support (20).

11. The method of claim 8, wherein steps (a), (b) and (c) are individually accomplished via different ingestible supports (20).

12. The method of claim 9, wherein the ingestible support (20) includes an illuminating module (30, 114, 322, 422, 452) for illuminating the targeted cell or tissue, a detecting module (40, 124, 324, 424, 434, 454) for detecting the targeted cell or tissue, an imaging module (50, 326, 432, 456) for imaging the targeted cell or tissue, and a control module (60) operatively connected to the illuminating module (30, 114, 322, 422, 452), the detecting module (40, 124, 324, 424, 434, 454), and the imaging module (50, 326, 432, 456).

13. The method of claim 12, wherein the control module (60) controls the operation of the illuminating module (30, 114, 322, 422, 452), the detecting module (40, 124, 324, 424, 434, 454), and the imaging module (50, 326, 432, 456).

14. The method of claim 12, wherein the control module (60) controls the position and orientation of the support (20).

15. A system (10, 100, 300, 400) comprising:
at least one support (20);
at least one reservoir (70, 132, 312, 412, 442) suitable for retaining one or more nanoshells;
at least one delivery mechanism operatively associated with the reservoir (70, 132, 312, 412, 442) and suitable for delivering one or more nanoshells;
at least one imaging mechanism; and
a control mechanism operatively associated with each delivery mechanism and each imaging mechanism.

16. The system (10, 100, 300, 400) of claim 15, wherein one or more supports (20) are an ingestible capsule.

17. The system (10, 100, 300, 400) of claim 16, wherein a reservoir (70, 132, 312, 412, 442) and a delivery mechanism are included as part of one ingestible capsule.

18. The system (10, 100, 300, 400) of claim 17, wherein the imaging mechanism is included as part of an additional ingestible capsule.

19. The system (10, 100, 300, 400) of claim 17, wherein an additional reservoir (70, 132, 312, 412, 442) and delivery mechanism for retaining and delivering one or more additional nanoshells are included as part of the ingestible capsule.

20. The system (10, 100, 300, 400) of claim 16, wherein a reservoir (70, 132, 312, 412, 442), a delivery mechanism and an imaging mechanism are included as part of one ingestible capsule.
